(19) United States
(12) Patent Application Publication
(10) Pub. No.: US 2025/0040578 A1
(43) Pub. Date: Feb. 6, 2025

(54) SCAFFOLD FOR CULTURED MEAT THAT ENHANCES THE MEAT FLAVOR OF CULTURED MEAT

(71) Applicant: UIF (UNIVERSITY INDUSTRY FOUNDATION), YONSEI UNIVERSITY, Seoul (KR)

(72) Inventors: Jinkee HONG, Seoul (KR); Woojin CHOI, Seoul (KR); Milae LEE, Seoul (KR)

(73) Assignee: UIF (UNIVERSITY INDUSTRY FOUNDATION), YONSEI UNIVERSITY, Seoul (KR)

(21) Appl. No.: 18/765,590

(22) Filed: Jul. 8, 2024

(30) Foreign Application Priority Data
Jul. 7, 2023 (KR) 10-2023-0088104
Jul. 4, 2024 (KR) 10-2024-0088044

(51) Int. Cl. A23L 13/00; A23L 27/20; A23L 29/281; C12N 5/071

(52) U.S. Cl. CPC A23L 13/00; A23L 27/2022; A23L 27/2052; A23L 27/2054; A23L 29/284; C12N 5/0697; C12N 2533/30; C12N 2533/50; C12N 2537/10

(57) ABSTRACT

The present disclosure is directed to providing a scaffold for cultured meat that can enhance the flavor and taste of cultured meat economically and can provide cultured meat with a taste similar to that of real meat. Cultured meat prepared according to the method for preparing cultured meat of the present disclosure can provide excellent flavors such as savoriness, meaty flavor, roasted meaty flavor, salty flavor, etc.

### CROSS-REFERENCE TO RELATED APPLICATION

This application claims priority to Korean Patent Application Nos. 10-2023-0088104 and 10-2024-0088044 filed on Jul. 7, 2023 and Jul. 4, 2024, respectively, and all the benefits accruing therefrom under 35 U.S.C. § 119, the contents of which in its entirety are herein incorporated by reference.

### BACKGROUND

### 1. Field

The present disclosure relates to a scaffold for cultured meat capable of producing cultured meat with enhanced meaty flavor, a method for preparing cultured meat using the same, and a food composition containing the cultured meat.

### 2. Description of the Related Art

Due to abnormal weather such as warming, crop yields are decreasing and the production cost of feed grains is increasing. As a result, livestock products are becoming more expensive. Animal-derived foods such as livestock products are the best source of high-quality proteins and micronutrients, and demand is expected to increase further as they contain essential amino acids that are not synthesized in the body and must be consumed through food.

Therefore, the development of plant-based meat and cell-cultured meat as safe and sustainable alternative protein sources to replace animal meat has become active recently. Cultured meat refers to edible meat obtained through cell proliferation by cell engineering technology by cultivating cells of living animals in a laboratory without going through the process of rearing livestock. It is also called in-vitro meat or lab-grown meat in that it is grown in a test tube, artificial meat in that it is synthesized using human stem cells, clean meat in that it is produced in a clean production facility rather than a traditional rearing facility, and bio-artificial muscles in that muscle fibers (BAMs) that constituting muscles are cultivated. However, for commercialization of the cultured meat, it is important not only to increase the nutritional value of the cultured meat through production of high-quality proteins but also to ultimately preserve the taste of the meat. However, because foods made with proteins only do not have enough factors for enjoying the taste, research is being actively conducted on the color, marbling, texture, flavor, etc. of cultured meat.

Since the research on cultured meat is still at the cell culture scale rather than tissue culture level, there are limitations in replicating the sensory characteristics of meat. In particular, the flavor ingredients of meat, such as aldehydes, alcohols, sulfur-containing compounds, etc. generated during cooking, are important. These flavor compounds are produced when peptides react with reducing sugars at high temperatures (over 150° C.). This phenomenon is called the Maillard reaction. Various Maillard reaction products are produced depending on the type of amino acids and the amount of proteins and sugars, and the overall taste of the cooked meat is determined by them. The difference in amino acid profiles between in-vitro tissue and real meat makes it difficult to mimic the Maillard flavor of real meat in the field of cultured meat. Therefore, in order to increase the sensory similarity between cultured meat and real meat, it is necessary to mimic the Maillard flavor in cultured meat.

Recently, researches on the improvement of the flavor characteristics of cultured meat have been reported. For example, a research comparing the taste of cultured muscle tissue obtained from chicken satellite cells with the taste of regular chicken meat has been reported. However, because the amino acid composition is different, it is still difficult to mimic the taste of real meat in tissue cultured without any other treatment.

### REFERENCES OF RELATED ART

### Patent Documents

Korean Patent Publication No. 10-2022-0040424.

### SUMMARY

The present disclosure is directed to providing a scaffold for cultured meat that can improve the flavor of cultured meat and produce cultured meat with a taste similar to that of real meat.

The present disclosure is also directed to providing a scaffold for cultured meat that can enhance the flavor of cultured meat economically and a method for preparing cultured meat using the same.

According to an aspect of the present disclosure, there is provided a scaffold for cultured meat, containing a hydrogel to which a flavor compound containing a disulfide bond-containing functional group is bound.

Specifically, the disulfide bond-containing functional group may be represented by any one selected from Chemical Formulas 1 to 3.

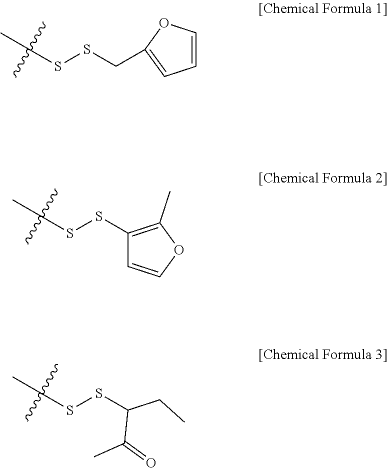

More specifically, the flavor compound may be represented by Chemical Formula 4.

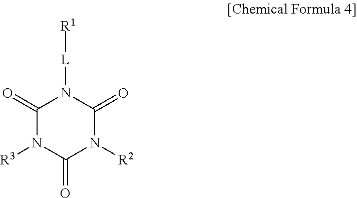

In Chemical Formula 4,
R¹ is any one of the functional groups represented by Chemical Formulas 1 to 3,
L is (C₁-C₃₀) alkylene, (C₂-C₃₀) heteroalkylene, (C₃-C₃₀) cycloalkylene, (C₂-C₃₀) heterocycloalkylene, (C₆-C₃₀) arylene or (C₅-C₃₀) heteroarylene,
the (C₂-C₃₀) heteroalkylene is one in which each of at least one of a plurality of methylenes (—CH₂—) is independently substituted with —O—, —NH— or —C(═O)—, and
each of R² and R³ is independently a substituent having an acrylic group or a methacryl group bonded to a terminal.

Further more specifically, Chemical Formula 4 may be represented by Chemical Formula 5.

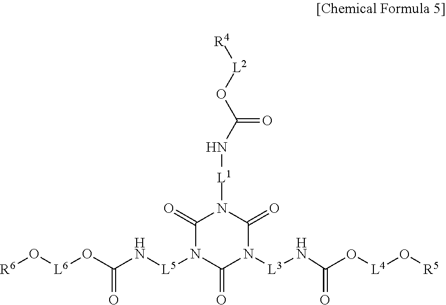

In Chemical Formula 5,
R⁴ is any one of the functional groups represented by Chemical Formulas 1 to 3, each of R⁵ and R⁶ are is an acrylic group or a methacrylic group,
L₁ is (C₁-C₁₅) alkylene,
L₂ is (C₁-C₅) alkylene,
each of L₃ and L₅ is independently (C₁-C₁₅) alkylene, and
each of L₄ and L₆ is independently (C₁-C₅) alkylene.

Most specifically, in Chemical Formula 5,
R⁴ is any one of the functional groups represented by Chemical Formulas 1 to 3,
R⁵ and R⁶ are methacryl groups,
L₁ is (C₄-C₈) straight-chain alkylene,
L₂ is (C₂-C₄) straight-chain alkylene,
each of L₃ and L₅ is independently (C₄-C₈) straight-chain alkylene, and
each of L₄ and L₆ is independently (C₂-C₄) straight-chain alkylene.

The hydrogel may include a protein-based polymer.

The acrylic or methacrylic group of the flavor compound may be copolymerized with the acrylic or methacrylic group side chain of the protein-based polymer to form a hydrogel.

The flavor compound may be contained in an amount of 0.1 to 5 wt % based on the total weight of the hydrogel on a dry mass basis.

According to another aspect of the present disclosure, there is provided a method for preparing cultured meat, which includes: a step of filling a scaffold for cultured meat with a culture medium and inoculating animal cells; a step of incubating the culture medium inoculated with the animal cells to proliferate the cells; and a step of organizing the animal cells proliferated in the culture medium.

The animal cells may include animal myoblasts and animal adipocytes.

The organization step may involve treating the animal cells with stimulus such as ultrasound, electric current, electromagnetic field, magnetic field or a combination thereof.

According to another aspect of the present disclosure, there is provided cultured meat prepared by the method for preparing cultured meat.

According to another aspect of the present disclosure, there is provided a food composition containing the cultured meat.

The scaffold for cultured meat according to an exemplary embodiment of the present disclosure can release meaty flavor at high temperature and can enhance meaty flavor in cultured meat economically.

Cultured meat prepared according to the method for preparing cultured meat of the present disclosure can provide excellent flavors such as savoriness, meaty flavor, roasted meaty flavor, salty flavor, etc.

### BRIEF DESCRIPTION OF THE DRAWINGS

FIG. 1 shows schematic illustration of a switchable flavor system and classification of flavor compounds.

FIG. 2 shows chemical structure and 1H NMR analysis results for switchable flavor compounds (SFCs) prepared in Preparation Examples 1 to 3.

FIGS. 3A, 3B and 3C show the structure of switchable flavor compounds (SFCs) and ultraviolet-visible (UV-Vis) spectra obtained by heating the SFCs in Test Example 1.

FIG. 4 shows a result of measuring the stability of switchable flavor compound (SFC) itself in Test Example 1.

FIG. 5 shows a result of evaluating the flavor stability of a hydrogel scaffold in Test Example 1.

FIG. 6 shows the flavor enriching process of a switchable flavor compound (SFC) in a cultured meat preparation process according to Test Example 2.

FIGS. 7A and 7B shows a result of measuring the viability of cells cultured on a scaffold in Test Example 2.

FIG. 8 shows a result of measuring the degree of differentiation of cells on a scaffold in Test Example 2.

FIG. 9 shows a result of comparing the flavor profiles of scaffolds in Test Example 2.

FIG. 10 is shows schematic illustration of cultured meat samples used in electronic nose analysis in Test Example 3.

FIG. 11 shows a result of analyzing flavor compounds of cultured meat in Test Example 3.

FIG. 12 shows a result of principal component analysis (PCA) for analysis of flavor similarity between cultured meat groups in Test Example 3.

### DETAILED DESCRIPTION

The present disclosure is described in detail below. Unless defined otherwise, the terms used in this specification should be construed as generally understood by those who have ordinary knowledge in the art. The drawings and exemplary embodiments of this specification are intended to make it easy for those having ordinary skill to understand and implement the present disclosure. The contents that may obscure the gist of the present disclosure may be omitted from the drawings and exemplary embodiments, and the present disclosure is not limited to the drawings and exemplary embodiments.

As used herein, singular forms are intended to include plural forms as well, unless the context clearly dictates otherwise.

Numerical ranges used herein include the lower and upper limits and all values within the ranges, increments logically derived from the ranges being defined, all doubly defined values, and all possible combinations of the upper and lower limits of the numerical ranges defined in different forms. Unless defined otherwise in this specification, values outside the numerical ranges that may occur due to experimental errors or rounding of values are also included in the defined numerical ranges.

The terms such as include, have, possess, etc. used in this specification indicate the existence of the features or components described in the specification and do not preclude the possibility of the addition of one or more other features or components, unless specified otherwise.

In this specification, “alkyl group” means an aliphatic hydrocarbon group, unless defined otherwise.

The alkyl group may be a “saturated alkyl group” that does not contain any double or triple bond.

The alkyl group may also be an “unsaturated alkyl group” containing at least one double or triple bond.

The alkyl group, whether saturated or unsaturated, may be branched, straight-chain or cyclic.

The alkyl group may be a C₁-C₃₀ alkyl group. More specifically, it may be a C₁-C₂₀ alkyl group, a C₁-C₁₀ alkyl group or a C₁-C₆ alkyl group.

For example, a C₁-C₄ alkyl group having 1 to 4 carbon atoms in the alkyl chain is selected from methyl, ethyl, propyl, isopropyl, n-butyl, isobutyl, sec-butyl and t-butyl.

As specific examples, the alkyl group includes a methyl group, an ethyl group, a propyl group, an isopropyl group, a butyl group, an isobutyl group, a t-butyl group, a pentyl group, a hexyl group, an ethenyl group, a propenyl group, a butenyl group, a cyclopropyl group, a cyclobutyl group, a cyclopentyl group, a cyclohexyl group, etc.

A “cycloalkyl group” includes a monocyclic or fused-ring polycyclic (i.e., rings sharing adjacent carbon atoms) functional group.

A “heterocycloalkyl group” means a cycloalkyl group containing 1 to 4 heteroatoms selected from a group consisting of N, O, S and P, with the remainder being carbon. When the heterocycloalkyl group is a fused ring, at least one ring of the fused ring may contain 1 to 4 heteroatoms.

An “aryl group” include a monocyclic or fused ring polycyclic (i.e., rings sharing adjacent carbon atom) functional group.

A “heteroaryl group” means an aryl group containing one or more heteroatoms selected from a group consisting of N, O, S and P, with the remainder being carbon. When the heteroaryl group is a fused ring, at least one ring of the fused ring may contain 1 to 4 heteroatoms.

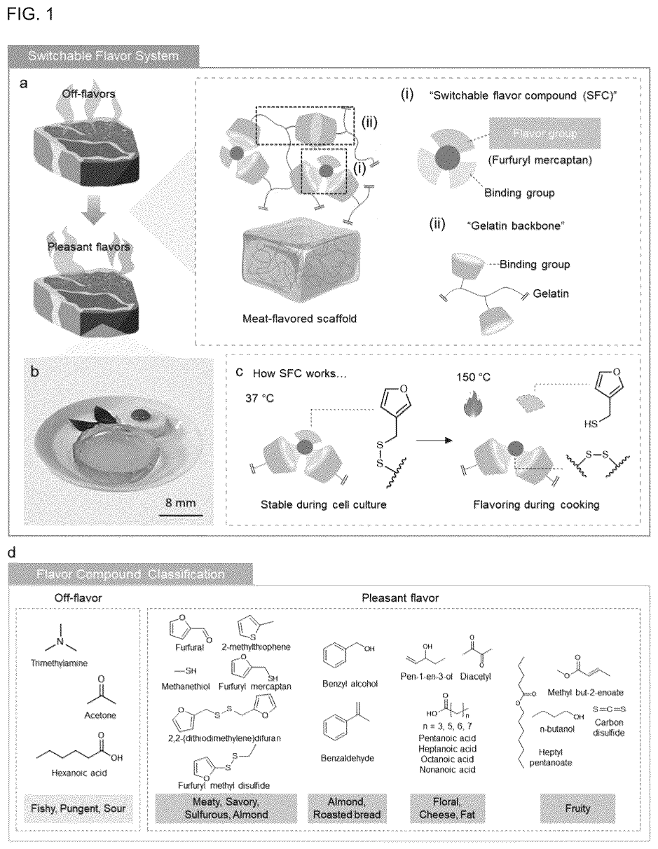

FIG. 1 shows schematic illustration of a switchable flavor system and classification of flavor compounds. Hereinafter, the scaffold for cultured meat of the present disclosure and the method for preparing cultured meat using the same will be described with reference to FIG. 1.

The present disclosure relates to a scaffold for cultured meat that can enhance the flavor of cultured meat and a method for preparing cultured meat using the same.

A scaffold for cultured meat is a structure on which animal cells, which become the source cells of cultured meat, can attach and grow. When animal cells are inoculated into a medium containing a scaffold for cultured meat, the animal cells attach to the micropores of the scaffold, and the culture medium flows in and out through other micropores to supply nutrients to the cells. The micropores inside a conventional scaffold for cultured meat are advantageous for cell growth, but they have limitations in improving the taste of meat because it is difficult to obtain cultured meat full of meat ingredients. Accordingly, the present disclosure provides a scaffold for cultured meat that can improve the taste of cultured meat economically without improving the ingredients of cultured meat themselves.

The scaffold for cultured meat of the present disclosure contains a hydrogel to which a flavor compound containing a disulfide bond-containing functional group is bound.

According to an exemplary embodiment of the present disclosure, the disulfide bond-containing functional group may be represented by any one selected from Chemical Formulas 1 to 3.

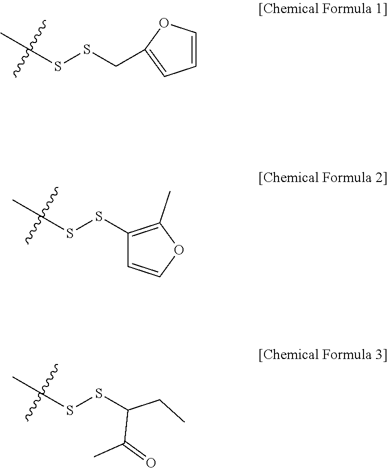

The scaffold for cultured meat of the present disclosure is integrated with cultured meat prepared using the scaffold for cultured meat, and as a part of the cultured meat, it can enhance the meaty flavor of the cultured meat during cooking.

Specifically, if the scaffold for cultured meat of the present disclosure is heated at high temperature of 100° C. or higher, the disulfide bond of the flavor compound contained in the scaffold for cultured meat is cleaved, and furfuryl mercaptan, which features meaty flavor, is released outside of the scaffold, providing meaty aroma and flavor. Accordingly, the cultured meat prepared using the scaffold for cultured meat can have excellent meaty taste, such as meaty flavor, roasted meaty flavor, salty taste, etc., due to furfuryl mercaptan released from the scaffold during cooking.

According to another exemplary embodiment of the present disclosure, the flavor compound may be represented by Chemical Formula 4.

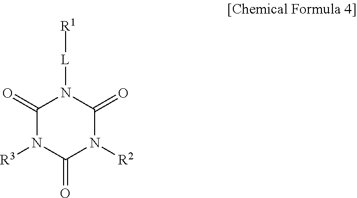

In Chemical Formula 4,
R¹ is any one of the functional groups represented by Chemical Formulas 1 to 3,
L is (C₁-C₃₀) alkylene, (C₂-C₃₀) heteroalkylene, (C₃-C₃₀) cycloalkylene, (C₂-C₃₀) heterocycloalkylene, (C₆-C₃₀) arylene or (C₅-C₃₀) heteroarylene,
the (C₂-C₃₀) heteroalkylene is one in which each of at least one of a plurality of methylenes (—CH₂—) is independently substituted with —O—, —NH— or —C(═O)—, and
each of R² and R³ is independently a substituent having an acrylic group or a methacryl group bonded to a terminal.

Here, R¹ is a functional group that imparts flavor to cultured meat, and R² and R³ are functional groups that bind to the hydrogel.

Specifically, Formula 4 may be represented by Chemical Formula 5.

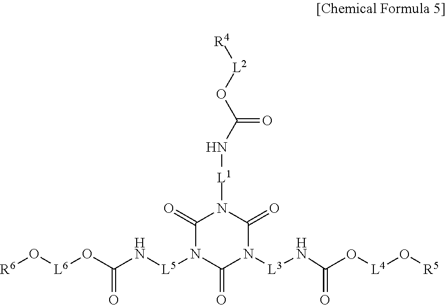

In Chemical Formula 5,
R⁴ is any one of the functional groups represented by Chemical Formulas 1 to 3,
each of R⁵ and R⁶ are is an acrylic group or a methacrylic group,
L₁ is (C₁-C₁₅) alkylene,
L₂ is (C₁-C₅) alkylene,
each of L₃ and L₅ is independently (C₁-C₁₅) alkylene, and
each of L₄ and L₆ is independently (C₁-C₅) alkylene.

More specifically, in Chemical Formula 5,
R⁴ is any one of the functional groups represented by Chemical Formulas 1 to 3,
R⁵ and R⁶ are methacryl groups,
L₁ is (C₄-C₈) straight-chain alkylene,
L₂ is (C₂-C₄) straight-chain alkylene,
each of L₃ and L₅ is independently (C₄-C₈) straight-chain alkylene, and
each of L₄ and L₆ is independently (C₂-C₄) straight-chain alkylene.

The hydrogel may include a protein-based polymer. The protein-based polymer may be cross-linked to secure a large surface area, and is advantageous for introducing various substances into the scaffold.

Specifically, the protein-based polymer may be a combination of one or more selected from silk, fibrinogen, fibrin, thrombin, collagen, elastin, albumin, gelatin, keratin, laminin and methacrylated polymer materials thereof. The protein-based polymer may have some amino acid residues substituted with a polymerizable functional group, and specifically may have a structure in which a polymerizable functional group is introduced into the side chain of the protein-based polymer. The polymerizable functional group is not limited as long as it is a functional group that can be polymerized through radical polymerization, and may be, for example, a (meth)acrylic group or a vinyl group. According to one non-limiting exemplary embodiment, the protein-based polymer may be gelatin methacryloyl (GelMA).

The (meth)acrylic group of the flavor compound may be copolymerized with the (meth)acrylic group side chain of the protein-based polymer to form a hydrogel.

According to an exemplary embodiment, the scaffold for cultured meat may contain 0.1 to 5 wt %, specifically 0.1 to 3 wt %, more specifically 0.1 to 1 wt %, of the flavor compound based on the total weight of the hydrogel on a dry mass basis.

The method for preparing cultured meat according to the present disclosure may include: a step of filling a scaffold for cultured meat with a culture medium and inoculating animal cells; a step of incubating the culture medium inoculated with the animal cells to proliferate the cells; and a step of organizing the animal cells proliferated in the culture medium.

The culture medium may contain a number of growth factors and nutrients, specifically one or more selected from epithelial growth factor (EGF), insulin-like growth factor (IGF-1), platelet-derived growth factor (PDGF), transforming growth factor-beta (TGF-β), vascular endothelial growth factor (VEGF), leukemia inhibitory factor (LIF) and basic fibroblast growth factor (bFGF), although not being limited thereto.

The scaffold for cultured meat is a three-dimensional scaffold for the proliferation of animal cells, and provides a biological environment in which cell attachment, proliferation and differentiation can occur well by mimicking the various roles of the extracellular matrix in a given environment. The animal cells can bind to the micropores of the scaffold for cultured meat and proliferate as integrated with the scaffold for cultured meat.

The animal cells may include animal stem cells and animal adipocytes, and are not limited as long as they are cells that can be used to produce cultured meat.

The animal stem cells may be stem cells derived from non-human mammals, birds, reptiles, fish, crustaceans or molluscs, and may specifically be one or more selected from mesenchymal stem cells (MSCs), induced pluripotent stem cells (iPSCs), satellite cells, adipose-derived stem cells (ASCs) and embryonic stem cells. The stem cells may be obtained by collecting tissue from a living animal and then isolating stem cells from the tissue.

The organization step may involve treating the animal cells with stimulus such as ultrasound, electric current, electromagnetic field, magnetic field or a combination thereof. The stimulus is a physical stimulus including mechanical stimulus or electrical stimulus. By applying an appropriate physical stimulus, an environment similar to the actual body such as the circulatory system, nervous system and muscles, where various stimulations exist, can be created. Through this, cell growth may be promoted and the shape, function, and development of muscle cells may be regulated, allowing the organization step to occur.

In addition, the method for preparing cultured meat may further include a step of adding a colorant to color the muscle fibers, if necessary. The colorant refers to a compound that imparts color to food. Artificial colorants, natural colorants, natural extracts (e.g., beetroot extract, pomegranate fruit extract, cherry extract, carrot extract, red cabbage extract or red seaweed extract), modified natural extracts, natural juices (e.g. beetroot juice, pomegranate juice, cherry juice, carrot juice, red cabbage juice or red seaweed juice), modified natural juices, FD&C (Food Drug & Cosmetics) Red No. 3 (erythrosine), FD&C Green No. 3 (Fast Green FCF), FD&C Red No. 40 (Allura Red AC), FD&C Yellow No. 5 (tartrazine), FD&C Yellow No. 6 (Sunset Yellow FCF), FD&C Blue No. 1 (Brilliant Blue FCF), FD&C Blue No. 2 (indigotine), titanium oxide, annatto, anthocyanin, betanin, beta-APE 8 carotene, beta-carotene, black currant, burnt sugar, canthaxanthin, caramel, carmine/carminic acid, cochineal extract, curcumin, lutein, carotenoid, monascin, paprika, riboflavin, saffron, turmeric and combinations thereof can be used to reproduce the red color of beef or pork, although not being particularly limited thereto. Additionally, a colorant such as nitrite and a coloring aid that promotes color development of the nitrite colorant, such as ascorbic acid, erythorbic acid or salts thereof, may be further added.

In an exemplary embodiment of the present disclosure, the method for preparing cultured meat may further include a step of adding fat. Specifically, in the step of inoculating the animal cells, animal adipocytes may be injected together and co-cultured during the proliferation of muscle cells, or fat in liquid form may be added. In this case, it is advantageous for health because the saturated fatty acids contained in meat can be replaced with beneficial fats. Specifically, as the fat, vegetable oils such as soybean oil, corn oil, canola oil, rice bran oil, sesame oil, extracted sesame oil, perilla oil, extracted perilla oil, safflower oil, sunflower oil, cottonseed oil, peanut oil, olive oil, palm oil, coconut oil, red pepper seed oil, etc., animal fats and oils such as edible beef tallow, edible pork tallow, raw beef tallow, raw pork tallow, fish oil, etc., and edible oil and fat processed goods such as blended cooking oil, seasoning oil, processed oil and fat, shortening, margarine, imitation cheese, vegetable cream, etc. can be used.

The present disclosure provides cultured meat prepared by the method for preparing cultured meat described above. The cultured meat is integrated with the scaffold for cultured meat according to an exemplary embodiment of the present disclosure, and can enhance the meaty flavor of the cultured meat during cooking by releasing the flavor compound contained in the scaffold for cultured meat.

Additionally, the present disclosure provides a food composition containing the cultured meat. In an exemplary embodiment of the present disclosure, the food may be one or more selected from a group consisting of snacks, dumplings, fried foods, stir-fried foods, sauces, seasonings, powder mixes, breads, beverages, processed canned foods, dried seaweeds and and processed noodles. The cultured meat may be ground into various particle sizes depending on purposes for addition to the food. It can be ground uniformly or unevenly within a range of 1 μm to 10 cm for addition to the food.

Hereinafter, the scaffold for cultured meat according to the present disclosure and the cultured meat prepared using the same will be described in more detail through specific examples. However, the following examples are only examples for describing the present disclosure in detail, and the present disclosure can be implemented in various forms without being limited thereto. In addition, the terms used in the description of the present disclosure are intended only to effectively describe the specific examples and are not intended to limit the present disclosure.

### EXAMPLES

### Synthesis of SFC

### Preparation Example 1: Synthesis of Furfuryl Mercaptan-Based SFC

To induce the formation of disulfide bonds at the thiol groups of Maillard reaction products, 11.6 mmol of 3-mercapto-1-propanol (HS(CH₂CH₂CH₂)OH) was reacted with an equimolar amount of furfuryl mercaptan at 80° C. for 48 hours, along with 2.56 mmol of hydrogen peroxide (an oxidizing agent to form disulfide bonds). As a result, a furfuryl mercaptan-based disulfide-bonded Maillard reaction product was prepared.

A switchable flavor compound (SFC) was prepared via urethane reaction with an isocyanate trimer (hexamethylene diisocyanate isocyanurate trimer) represented by the Chemical Formula A. The molar ratio between the isocyanate trimer, 2-hydroxyethyl methacrylate and the disulfide-bonded Maillard reaction product was 1:2:1.

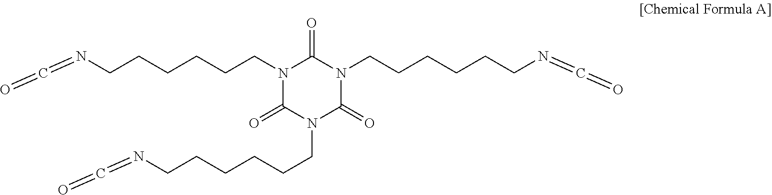 [SMILES: O=C=NCCCCCCn1c(=O)n(CCCCCCN=C=O)c(=O)n(CCCCCCN=C=O)c1=O]

The switchable flavor compound (SFC) based on furfuryl mercaptan was synthesized with a 30% (w/v) concentration of all reagents in propylene carbonate as a solvent. Specifically, a furfuryl mercaptan-based switchable flavor compound (SFC) was synthesized by reacting 0.5 mmol of isocyanate trimer (0.267 g) in 1 mL of propylene carbonate (1.2 g, 11.8 mmol) where the molar ratio of 2-hydroxyethyl methacrylate and disulfide-linked furfuryl mercaptan was set to 2 and 1, respectively, at 80° C. for 4 days.

### Preparation Example 2: Synthesis of 3-mercapto-2-pentanone-based SFC

A switchable flavor compound (SFC) was prepared using 3-mercapto-2-pentanone instead of furfuryl mercaptan as a flavor ingredient. Specifically, a 3-mercapto-2-pentanone-based switchable flavor compound (SFC) was synthesized in the same manner as in Preparation Example 1, except that 0.3 mmol of the isocyanate trimer was used instead of 0.5 mmol and reaction was performed at 80° C. for 3 days at a concentration of 20% (w/v) with the molar ratio of the isocyanate trimer, 3-mercapto-1-propanol and the disulfide-bonded Maillard reaction product set to 1:2:1.

### Preparation Example 3: Synthesis of 2-methyl-3-furanthiol-based SFC

A 2-methyl-3-furanthiol-based switchable flavor compound (SFC) was synthesized under the same conditions as in Preparation Example 2, except that 2-methyl-3-furanthiol was used instead of 3-mercapto-2-pentanone as the flavor ingredient.

### Confirmation of Synthesis of SFC

Chemical structure was investigated using Raman spectroscopy (XploRA PLUS, HORIBA, France) and ¹H NMR (Avance III HD 300, Bruker Biospin, USA). A 10× objective lens and 532 nm laser (75 mW intensity) were used for the Raman spectroscopy measurement. The peak at 520 cm⁻¹ was used as the calibration standard. The laser was irradiated for 30 seconds per cycle, and each analysis was repeated five times to acquire reliable spectra. For the NMR analysis, the switchable flavor compound (SFC) was dispersed in dimethyl sulfoxide-d₆ with the volume concentration of 10%.

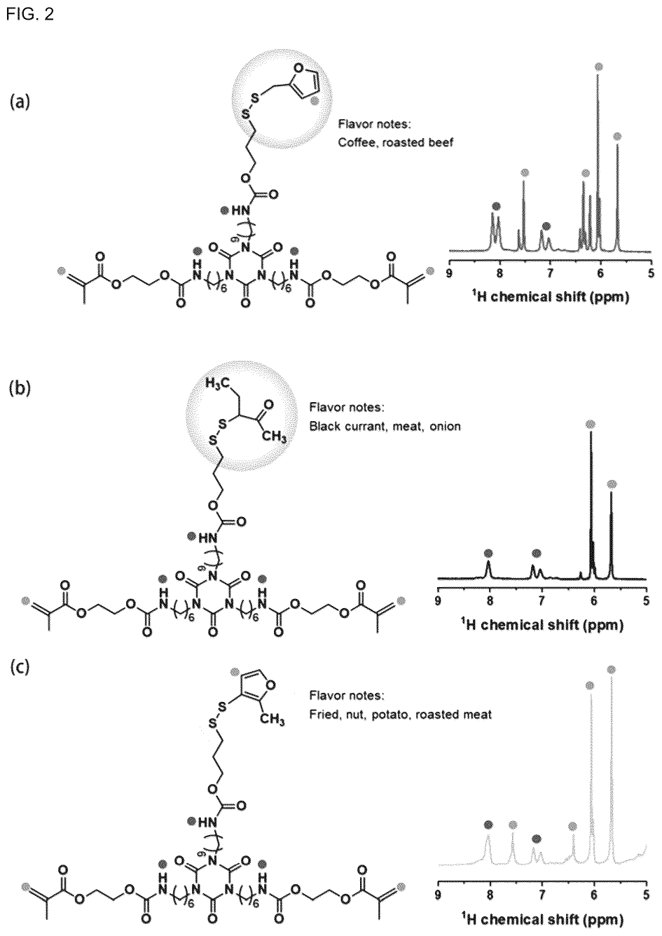

The chemical structure and ¹H NMR analysis results for the switchable flavor compounds (SFCs) of Preparation Examples 1 (a), 2 (b) and 3 (c) are shown in FIG. 2.

### Synthesis of Hydrogel Scaffold

### Example 1: Preparation of furfuryl mercaptan-based SFC-Conjugated Hydrogel (Gel+SFC) Scaffold

(1) Preparation of gelatin methacryloyl (GelMA)

First, gelatin methacryloyl (GelMA) was synthesized by conjugating methacrylic anhydride to fish gelatin. Specifically, fish gelatin (GELTECH, Korea) was dissolved in deionized water (DW) at 65° C. to make a 20% (w/v) gelatin solution. Then, 0.08 mL of methacrylic anhydride (Sigma-Aldrich #276685) was added to the gelatin solution per 1 g of fish gelatin. After stirring at 500 rpm for 2 hours, the gelatin solution was diluted 2-fold with distilled water. Then, the GelMA solution was dialyzed on a hot plate at 80° C. for 5 days using a 12-14 kDa membrane (Thermo Fisher, #08667E). Distilled water was used as the dialysate. The dialyzed GelMA solution was lyophilized for 5 days after 1 day of freezing at −20° C.

### (2) Preparation of Scaffold Precursor

After dissolving the lyophilized GelMA in deionized water at 50° C. to make a 20% (w/v) solution, a scaffold precursor was prepared by adding 0.1% (w/v) of 2-hydroxy-4-(2-hydroxyethoxy)-2-methylpropiophenone (12959; Sigma-Aldrich, #410896), which is a photoinitiator, and 0.5% (w/v) of the furfuryl mercaptan-based SFC of Preparation Example 1.

### (3) Preparation of Scaffold by Photopolymerization

The hydrogel precursor was distributed into a 24-well plate (SPL) and then exposed to ultraviolet light (UV) for 3 hours to complete polymerization. The hydrogel was washed 4 times with distilled water to remove unreacted molecules. The first two times of washing were performed by immersing the hydrogel in water for 1 minute for each wash. Then, the hydrogel was immersed in water at 37° C. for 6 hours to allow the hydrogel to swell. Afterwards, the hydrogel was taken out of the water and washed with fresh distilled water for 1 minute to obtain an SFC-conjugated hydrogel scaffold (Gel+SFC).

### Example 2: Preparation of 3-mercapto-2-pentanone-based SFC-Conjugated Hydrogel Scaffold (Gel+SFC)

An SFC-conjugated hydrogel scaffold (Gel+SFC) was prepared under the same condition as in Example 1, except that the 3-mercapto-2-pentanone-based SFC of Preparation Example 2 was used instead of the furfuryl mercaptan-based SFC of Preparation Example 1 in (2) Preparation of scaffold precursor.

### Example 3: Preparation of 2-methyl-3-furanthiol-based SFC-Conjugated Hydrogel Scaffold (Gel+SFC)

An SFC-conjugated hydrogel scaffold (Gel+SFC) was prepared under the same condition as in Example 1, except that the 2-methyl-3-furanthiol-based SFC of Preparation Example 3 was used instead of the furfuryl mercaptan-based SFC of Preparation Example 1 in (2) Preparation of scaffold precursor.

### Example 4: Preparation of Multiple SFC-Conjugated Hydrogel Scaffold (Gel+VSFC)

A multiple SFC-conjugated hydrogel scaffold (Gel+VSFC) was prepared under the same condition as in Example 1, except that 0.16% (w/v) of the furfuryl mercaptan-based SFC, 0.16% (w/v) of the 3-mercapto-2-pentanone-based SFC and 0.16% (w/v) of the 2-methyl-3-furanthiol-based SFC were added instead of 0.5% (w/v) of the furfuryl mercaptan-based SFC alone in (2) Preparation of scaffold precursor.

### Comparative Example 1: Preparation of Hydrogel Scaffold without SFC (Gel-SFC)

A hydrogel scaffold without SFC (Gel-SFC) was prepared under the same condition as in Example 1, except that the furfuryl mercaptan-based SFC of Preparation Example 1 was not used in (2) Preparation of scaffold precursor.

### Comparative Example 2: Preparation of Furfuryl Mercaptan-Mixed Hydrogel Scaffold (Gel+FM)

A furfuryl mercaptan-mixed hydrogel scaffold (Gel+FM) was prepared under the same condition as in Example 1, except that the pure furfuryl mercaptan was used instead of the furfuryl mercaptan-based SFC of Preparation Example 1 in (2) Preparation of scaffold precursor.

### Preparation of Cultured Meat: Cell Culturing

### Example 5: Preparation of Cultured Meat (CM+SFC) Using Gel+SFC

Before culturing cells, the Gel+SFC scaffold prepared in Example 1 was frozen at −20° C. for 1 day and then liophilized at −50° C. for 4 days using a liophilizer to obtain the scaffold. Then, the liophilized aerogel was washed with 70% (v/v) ethanol and sterilized by UV irradiation for 2 hours. The sterilized gel was swollen in high-glucose Dulbecco's modified Eagle's medium (HG-DMEM; Gibco) at 37° C. before cell seeding.

Bovine myoblasts were subcultured on a TPP® tissue culture dish (Sigma-Aldrich) using a growth medium composted of HG-DMEM (Thermo Fisher Scientific) with 10% (v/v) fetal bovine serum (FBS; Welgene), 1% (v/v) penicillin-streptomycin-amphotericin (PS; Gibco® Life Technologies) and 5 ng/mL basic fibroblast growth factor (bFGF; Peprotech, Rocky Hill, NJ, USA). For the cell subculture, the cells were washed with 1×PBS (Gibco® Life Technologies, USA) and detached using 0.025% (v/v) trypsin EDTA (Welgene).

The myoblasts at passage 3 were seeded on the Gel+SFC scaffold of Example 1 at a density of 2×10⁴ cells/scaffold (16.6 mm²×0.8 mm). The growth medium was replaced every two days to supply nutrients to the cells during proliferation. After 7 days of cell proliferation, cell differentiation was induced for 8 days using a differentiation medium consisting of HG-DMEM. The HG-DMEM was supplemented with 5% (v/v) horse serum (Gibco™, Thermo Fischer Scientific) and 1% (v/v) PS, and the differentiation medium was replaced every two days to prepare cultured meat (CM+SFC) using the Gel+SFC scaffold.

### Example 6: Preparation of Cultured Meat (CM+SFCV) Using Gel+SFCV

Cultured meat (CM+SFCV) was prepared under the same condition as in Example 5, except that the Gel+SFCV scaffold of Example 4 was used instead of the Gel+SFC scaffold of Example 1.

### Comparative Example 3: Preparation of Cultured Meat (CM−SFC) Using Gel-SFC

Cultured meat (CM+SFCV) was prepared under the same condition as in Example 5, except that the Gel-SFC scaffold of Comparative Example 1 was used instead of the Gel+SFC scaffold of Example 1.

### TEST EXAMPLES

### Experimental Methods

### (1) Thermal Responsivity Analysis of SFC

A UV quartz cuvette (chamber volume 3.5 mL, path length 10 mm) was filled with 2 mL of 1.86 μM SFC. The SFC was heated at 37° C., 80° C. and 150° C. for 24 hours by maintaining the closed system after sealing with a polytetrafluoroethylene stopper. UV-VIS spectra were obtained within 200-600 nm for the desired heating time points with the settings of bandwidth 1.0 nm, scan speed 240 nm/min and data interval 1.0 nm. The peak at 335 nm was assigned to furan-related absorbance. Here, the three replication experiments (n=3) were performed for each heating temperature for reliability.

To investigate the long-term stability under an open system, vials (chamber volume 5 mL, 18×38 mm²) were filled with 1 mL of 0.37 mM SFC (n=3) and 1 mL of pure furfuryl mercaptan (n=3), respectively. The vials containing the SFC and furfuryl mercaptan were positioned at 37° C. for up to 2 weeks. In particular, to emulate the open system during the cell culture procedure, the vials were not closed with a cap with sufficient air exchange maintained. Then, weight variation was measured for specific time points. The ¹H NMR (Avance III HD 300, Bruker, Bruker Biospin, USA) spectra of the residual SFC were obtained by diluting in dimethyl sulfoxide-d₆ (10 vol %).

### (2) Stability Analysis of Flavor Molecules in Hydrogel

To assess the stability of the flavor molecules in the hydrogel scaffold, the three types of samples containing the SFC-conjugated hydrogel scaffold of Example 1 (Gel+SFC), the SFC-free hydrogel scaffold of Comparative Example 1 (Gel-SFC) and the furfuryl mercaptan-mixed hydrogel of Comparative Example 2 (Gel+FM) were immersed in distilled water for the whole cell culture period (15 days). Then, the hydrogel scaffold sample was transferred to a vial and stored at room temperature or heated at 150° C. for 5 minutes. A heating plate was used for temperature control. The heating plate was covered with aluminum foil and the temperature was adjusted. When the temperature reached the set point, the sample was placed on a foil-covered plate for 5 minutes.

Volatile and semi-volatile compounds were detected for each group using GC-MS (Agilent 8890 GC system-Agilent 5677B MSD, Agilent Technologies). In particular, carboxen/polydimethylsiloxane/divinylbenzene (CAR/PDMS/DVB) fibers were used to adsorb the volatile compounds from the samples by headspace solid-phase microextraction (HS-SPME). At this time, the groups that were left at room temperature were heated to 30° C. for 20 minutes, while the remaining groups were heated to 80° C. for 20 minutes using an autosampler (Agilent 7693, CombiPAL Sampler 80, Agilent) to allow volatile compounds to be adsorbed onto the fibers. Then, the samples were maintained at room temperature with the fibers for additional 40 minutes to complete adsorption of the compounds. Subsequently, the volatile compounds adsorbed onto the fibers were analyzed. Helium with 99.999% purity was used as a carrier gas with a split flow rate of 40 mL/min. Fibers were injected into the inlet at a temperature of 250° C. using the split (20:1) injection mode. The initial temperature of a column oven was 40° C. and the hold time was 5 minutes. Then, the temperature was raised to 240° C. at a rate of 4° C./min, and the set temperature was maintained for 20 minutes to analyze the volatile compounds. The GC column used in this experiment was DB-WAX (Agilent 123-7063). The temperature of the column oven was kept at 40° C. for 5 minutes and then increased to 240° C. at a rate of 4° C./min. Finally, the column oven temperature was kept at 240° C. for 20 minutes to analyze the volatile and semi-volatile compounds of each group.

### (3) Biological Assessment of Cell Proliferation and Differentiation

For cell proliferation analysis, myoblasts were stained with CCK-8 (D-Plus™ CCK cell viability assay kit, Dongin LS, Korea) according to the protocol provided with the kit. To confirm cell adhesion, immunostaining was performed as follows. The cells were fixed first with formalin solution and then washed thoroughly with 1×PBS. Then, a blocking solution composed of 2% (w/v) bovine serum albumin (BSA; Sigma-Aldrich), 0.3% (v/v) Triton X-100 (Triton™ X-100 solution; Sigma-Aldrich) and 10% (v/v) horse serum dissolved in 1×PBS were added to the cells overnight. The F-actin filaments of myoblasts were stained using a staining solution containing 0.165 μM Alexa Fluor 488™ phalloidin (A12379, Thermo Fisher Scientific) and 0.08 mg/mL DAPI (Thermo Fisher Scientific, #D9542) in a solvent composed of 1×PBS and 1% BSA. For myosin heavy chain staining, MHC antibody was diluted 100-fold using an MF 20 concentrate solution (MF 20, DSHB, ID: AB2147781) in a solvent composed of 10% (v/v) horse serum and 2% (w/v) BSA. The MHC antibody solution was then added to the cells for 2 hours at room temperature. Then, the samples were washed twice with 1×PBS and once with 0.025% Triton X-100. Subsequently, 0.005 mg/mL secondary antibodies (donkey anti-mouse Alexa Flour 594, Thermo Fischer, #A21203) dissolved in the same solvent as MF20 were added to the samples at room temperature for 1 hour, followed by washing. Confocal laser scanning microscopy (CLSM; LSM 980, Carl Zeiss) was used to observe the stained cells. MHC quantification was performed using a bovine myosin-1 (MYH1) ELISA kit (MyBioSource).

### (4) Flavor Analysis

GC-MS and an electronic nose (HERACLES-II-E-NOSE, Alpha MOS, France) were used for flavor analysis. To compare the aromatic notes of the cultured meat (CM-SFC) of Comparative Example 3 and the cultured meat (CM+SFC) of Example 5, GC-MS was used to quantify the ratio of flavor compounds in each group, using the same stability analysis method employed for the flavor molecules in the hydrogel. Specifically, CM-SFC cultured according to Comparative Example 3 and CM+SFC cultured according to Example 5 were placed in separate vials and heated at 150° C. for 5 minutes before GC-MS analysis. To prevent the compounds from leaking, each vial was sealed with Parafilm before heating. Then, HS-SPME fibers were placed in each vial and the temperature was set to 80° C. to adsorb both the volatile and semivolatile compounds. GC-MS analysis was performed using the fibers with the compounds adsorbed for each group, employing the same method used for the stability analysis.

Meanwhile, in order to compare the flavor of the cultured meat prepared in Examples 5 and 6 and Comparative Example 3 with the flavor of Hanwoo beef brisket, an electronic nose (e-nose) was used to investigate the flavor pattern of each group. Hanwoo beef brisket was purchased from SIR.LOIN, Korea. For precise comparison, 0.4 g of CM−SFC, CM+SFC, CM+SFCV, and Hanwoo beef brisket were placed in a 20 mL headspace vial and heated at 150° C. for 5 minutes. Then, the volatile and semivolatile compounds produced in the headspace of the vial were injected into the inlet of the e-nose at an injection speed of 125 μL/s. The injector temperature was 200° C. and the injection time was 45 seconds. For carrier gas supply, H₂ was used to supply the injector and the detector. Two independent chromatographic columns and two flame ionization detectors were used for compound detection. The MXT-5 GC metal capillary column (Restek) was used as a nonpolar column and the MXT-1701 GC metal capillary column (Restek) was used as a second column with medium polarity. For the detectors, FID (flame ionization detector) 1 (non-polar) and FID2 (slightly polar) were used. The detector temperature was 260° C. The detected compounds were analyzed using the Alpha Software (Alpha MOS, France).

### (5) Assignment of Flavor Profile

The specific flavor note was assigned to each detected volatile compound using the Flavor Ingredient Library of the Flavor and Extract Manufacturers Association (FEMA) database.

### (6) Statistical Analysis

The data are reported as mean±standard deviation (SD). Statistical analysis was performed with a significance level of 0.05 using one-way analysis of variance with the Tukey method via OriginPro 2018 software. All experiments were repeated three times independently.

### TEST EXAMPLES

### Test Example 1: Evaluation of Thermal Responsivity

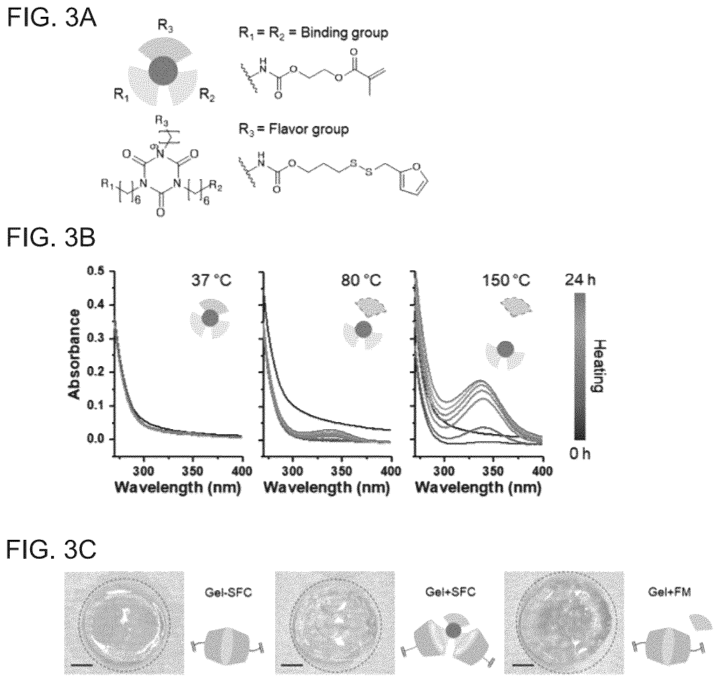

FIGS. 3A, 3B and 3C shows the structure of the switchable flavor compounds (SFCs) and ultraviolet-visible (UV-Vis) spectra obtained by heating the SFCs in Test Example 1.

Specifically, FIG. 3A shows the chemical structure of the switchable flavor compound (SFC) of Preparation Example 1, containing two binding groups (R₁-R₂) with methacrylate end and one flavor group (R₃) with a thermal responsive disulfide bridge. R₁ and R₂ are responsible for the robust bonds between the gelatin matrix, while R₃ can perform a temperature-responsive flavoring function. In the case of the thermal responsive group (R₃), a disulfide bond was introduced to furfuryl mercaptan, which is a roast meat-flavored Maillard reaction product. The disulfide bond can exhibit temperature responsivity through the disulfide exchange process.

FIG. 3B shows the ultraviolet-visible (UV-Vis) spectra of the SFC for monitoring the mobility of furfuryl mercaptan upon heating of the SFC. The peak at 335 nm indicates the mobility of the furan functional group of furfuryl mercaptan produced upon heating of the SFC. The intervals for heating were 0 minutes, 10 minutes, 1 hour, 4 hours, 7 hours, 12 hours and 24 hours. The temperature responsivity of the SFC was investigated by heating in a closed system at 37° C., 80° C. or 150° C. for up to 24 hours. When heated to 37° C., the SFC hardly yielded any noticeable signals. A weak absorbance signal started to appear after heating at 80° C. and this absorbance signal became obvious when heated to 150° C. In particular, the increasing peak around 335 nm indicated that the mobility of the furan group of furfuryl mercaptan, the Maillard reaction product, was improved. Hence, these results confirm that the SFC exhibited temperature-responsive flavoring via the release of the Maillard reaction product. The release of the Maillard reaction product exhibited a positive correlation with the heating temperature. Moreover, the onset temperature for flavoring was 80° C. and this thermo-responsivity became saturated after 12 hours.

FIG. 3C schematically shows the network structure of the SFC-free hydrogel scaffold (Gel-SFC) of Comparative Example 1, the SFC-conjugated hydrogel scaffold (Gel+SFC) of Example 1 and the furfuryl mercaptan-mixed hydrogel scaffold of Comparative Example 2 (Gel+FM) (scale bar: 0.4 cm). Gel+SFC features robust covalent bonds between the gelatin matrix and the SFC, while Gel+FM exhibits a weak interaction between the gelatin matrix and furfuryl mercaptan. The present disclosure is characterized in that a strong interaction is induced between SFC and the scaffold and then the SFC-derived Maillard reaction product is released selectively upon heating. Accordingly, the SFC was introduced into the gelatin-based hydrogel matrix based on the robust covalent bonds. In particular, the methacrylate of the SFC's binding group was connected with the methacrylate of gelatin methacryloyl through an ultraviolet (UV)-based radical reaction. In the furfuryl mercaptan-mixed hydrogel scaffold (Gel+FM) of Comparative Example 2, covalent bond was hardly found between the gelatin matrix and FM. The yellow color of Gel+FM is the due to the color of furfuryl mercaptan, indicating that it was not bonded to the gelatin matrix.

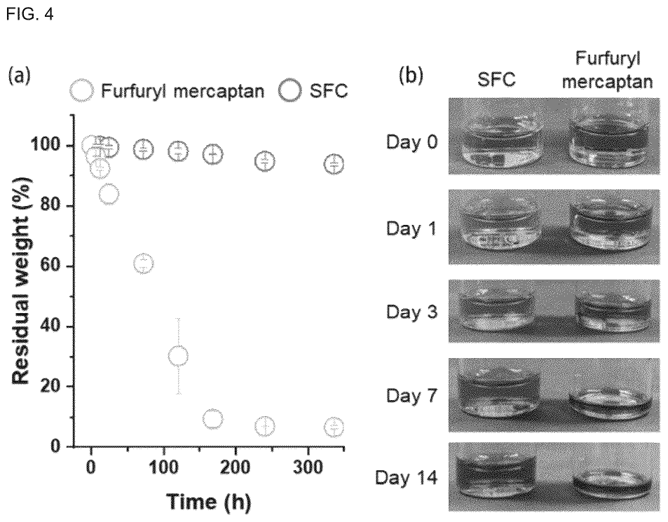

Moreover, in order to measure the stability of the switchable flavor compound (SFC) itself, the SFC of Preparation Example 1 and pure furfuryl mercaptan with no disulfide bond were residual weight and ¹H NMR analyses were performed at 37° C. for 14 days in an open system with sufficient air circulation, and the results are shown in FIG. 4. Pure furfuryl mercaptan evaporated rapidly from the first day of the experiment. In particular, the residual weight was only 60.9% and 6.76% after 3 and 14 days of incubation at 37° C., respectively. In contrast, the SFC of Preparation Example 1 maintained 93.8 wt % of its weight even at the same end point, and retained the same chemical structure. These results show that the SFC selectively released the flavor compound upon cooking and that the flavor compound was non-volatile during the cultured meat production. In other words, it can be seen that the SFC itself is a highly stable material.

Meanwhile, the flavor stability of the SFC-conjugated hydrogel scaffold (Gel+SFC) was evaluated.

The flavor stability of the SFC-conjugated hydrogel scaffold (Gel+SFC) of Example 1, the hydrogel scaffold without SFC (Gel-SFC) of Comparative Example 1 and the furfuryl mercaptan-mixed hydrogel scaffold (Gel+FM) of Comparative Example 2 at aqueous conditions was evaluated by gas chromatography-mass spectrometry (GC-MS) after immersing them in distilled water for 15 days, corresponding to the myoblast culture period.

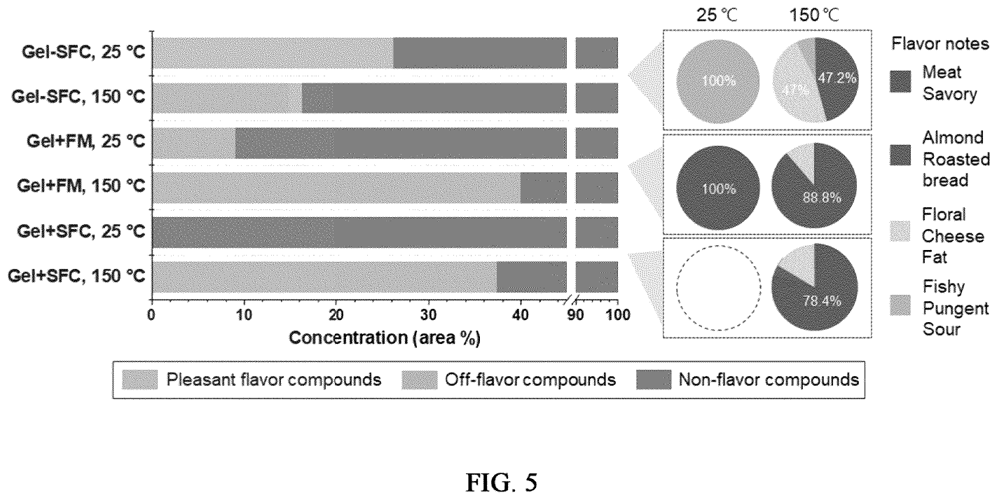

Specifically, to assess the flavor-releasing capability of the hydrogel scaffold as a function of temperature, the concentration of volatile compounds in each group at room temperature (25° C.) and at the Maillard reaction temperature (150° C.) was measured by GC-MS. The result is shown in FIG. 5. The flavor profiles of the detected volatile compounds were assigned by the Flavor Library of the FEMA (Flavor and Extract Manufacturer Association) database. Then, the volatile compounds with the flavor profiles assigned were classified into two groups: pleasant flavor and off-flavor. This classification was based on the flavor classification described in FIG. 1. The volatile compounds with no flavor profile were identified as non-flavor compounds. Also, the specific flavor notes that were identified from the FEMA library are shown in a pie chart.

For the hydrogel scaffold without SFC (Gel-SFC) of Comparative Example 1, the concentration of the compounds with off-flavor was decreased before heating, whereas the concentration of volatile compounds with pleasant flavor was increased after heating. Nevertheless, the off-flavor compounds still accounted for approximately 1.4% in Gel-SFC. Also, the concentration of the flavor compounds among total volatile compounds detected from Gel-SFC was decreased upon heating. For the SFC-conjugated hydrogel scaffold (Gel+SFC) of Example 1, the increase in the concentration of flavor compounds after heating was confirmed, which indicates that the introduction of the switchable flavor compound (SFC) can provide pleasant flavor properties to the scaffold. The temperature-responsive release of the flavor compounds was confirmed only in the SFC-conjugated hydrogel scaffold (Gel+SFC) of Example 1. At room temperature (25° C.), no flavor compound was detected in Gel+SFC. Upon heating at the Maillard reaction temperature (150° C.), high levels of the flavor compounds with pleasant flavor notes such as meat, savory, almond, roasted bread, floral, cheese and fat were detected from Gel+SFC. On the other hand, the release of the flavor compound before heating was detected for the furfuryl mercaptan-mixed hydrogel scaffold (Gel+FM) of Comparative Example 2, indicating that flavor loss can occur during the cell culture period. These results confirm that the switchable flavor compound (SFC) can contribute to the controlled release of the meaty flavor compounds from the scaffold, eventually enabling the production of flavor-rich cultured meat.

### Test Example 2: Biological Evaluation and Flavor Analysis of Cell-Cultured Scaffold

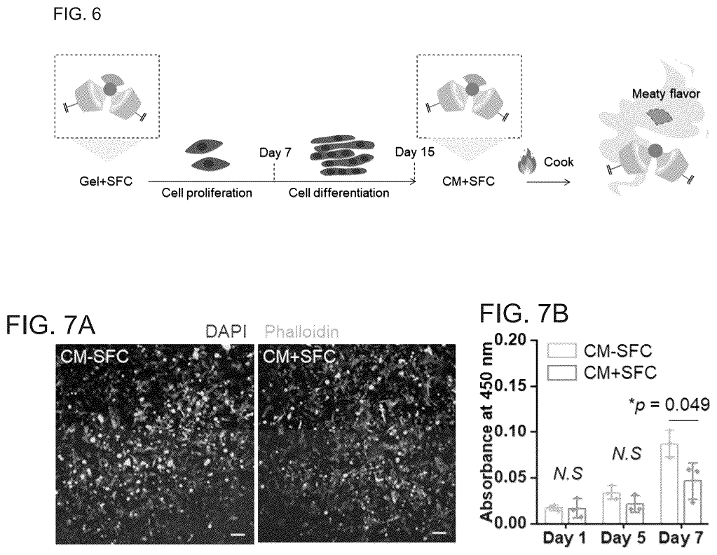

FIG. 6 shows the flavor enriching process of the switchable flavor compound (SFC) in the cultured meat preparation process. Specifically, the SFC-conjugated hydrogel scaffold (Gel+SFC) of Example 1 and the hydrogel scaffold without SFC (Gel-SFC) of Comparative Example 1 were lyophilized before cell culturing. Then, bovine myoblasts were cultured on the scaffold to assess the cell proliferation and differentiation behavior. After 15 days of cell culturing on the scaffold, cultured meat (CM) was prepared and cooked at the Maillard reaction temperature (150° C.) to volatilize the meaty flavor compounds of the switchable flavor compounds (SFC).

FIG. 7A shows the immunofluorescence images of myoblasts proliferated in cultured meat (CM−SFC) cultured in the Gel-SFC of Comparative Example 3 and cultured meat (CM+SFC) cultured in the Gel+SFC of Example 5 (scale bar: 100 μm), and FIG. 7B shows cell viability on days 1, 5 and 7 measured using the CCK-8 assay kit. CM−SFC was used as a control group to understand the influence of SFC on cell viability and myotube formation. First, immunostaining of actin filaments and nuclei of the cells was performed on day 7 of proliferation. The morphology and distribution of the attached cells was not significantly different between the scaffolds. To quantitatively compare the cell viability of the scaffolds, Cell Counting Kit-8 (CCK-8) assay was performed for CM-SFC and CM+SFC on days 1, 5 and 7 of proliferation. According to this, the cell survival rate on day 7 was lower in CM+SFC as compared to CM−SFC. However, the absorbance value did not decrease from day 1 to day 7 in CM+SFC. These results indicate that, rather than cell death in CM−SFC, the proliferation rate was slower in CM+SFC.

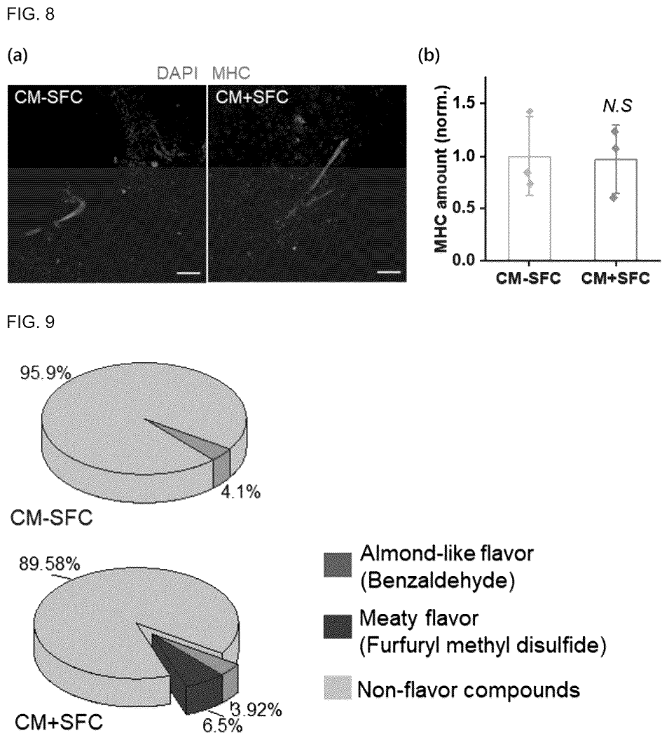

To investigate whether the slower cell growth in the SFC-conjugated hydrogel scaffold (Gel+SFC) of Example 1 was due to the flavor compound, differentiation was induced by replacing the growth medium with a differentiation medium. FIG. 8(a) shows the confocal images showing myosin heavy chain (MHC) and nuclei immunostained with MF20 (red) and DAPI (blue), respectively (scale bar: 100 μm), and FIG. 8(b) shows the result of quantitative assessment of the amount of myosin heavy chain (MHC) by enzyme-linked immunosorbent assay (ELISA) of bovine myosin-1. The MHC amount of the cultured meat (CM+SFC) of Example 5 was normalized to the MHC amount of the cultured meat (CM−SFC) of Comparative Example 3. Myogenic differentiation was induced on day 7 of proliferation and continued for the next 8 days. On the last day of myogenesis, myosin heavy chain (MHC) immunostaining was performed to compare the degree of myotube formation in the cultured meat of Example 5 (CM+SFC) and the cultured meat of Comparative Example 3 (CM−SFC). Branched myotubes were observed in both samples. Furthermore, the amount of MHC per scaffold was quantified by bovine myosin-1 enzyme-linked immunosorbent assay (ELISA), which revealed similar levels of MHC in CM−SFC and CM+FC. These biological test results indicate that the introduction of SFC into the scaffold does not inhibit cell functions.

FIG. 9 shows the result of assessing the volatile compounds in CM−SFC and CM+SFC after heating at 150° C. to compare the flavor profiles after myogenic differentiation for the different scaffolds. Only benzaldehyde with almond-like flavor was detected from the cultured meat (CM−SFC) of Comparative Example 3. On the other hand, meaty flavor was confirmed in the cultured meat (CM+SFC) of Example 5, due to the formation of furfuryl methyl disulfide derived from the volatilization of the complex flavor compounds. These results indicate that SFC can provide meat-like flavor to cultured meat.

### Test Example 3: Electronic Nose Analysis of Cultured Meat

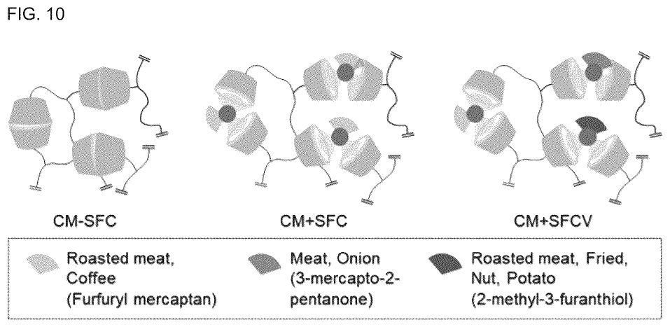

FIG. 10 is a schematic diagram of cultured meat samples used in electronic nose analysis. The cultured meat of Comparative Example 3 cultured on a scaffold without SFC (CM−SFC), the cultured meat of Example 5 cultured on a scaffold with SFC (CM+SFC), and the cultured meat of Example 6 cultured on a scaffold with multiple SFCs (CM+SFCV) are illustrated schematically. It was confirmed that the cultured meat cultured on a scaffold containing SFC stably released the Maillard flavor compounds upon heating. In slaughtered meat, a variety of Maillard reaction products with different flavor profiles are formed, rather than a single flavor compound. Therefore, the range of SFC was expanded with three different Maillard reaction molecules to verify if the SFC system can be applied not only to a single flavor compound but also to various flavor compounds. The thiol-ends of 3-mercapto-2-pentanone and 2-methyl-3-furanthiol may contribute to the formation of the thermo-responsive disulfide linkage, identically to the thiol-end of furfuryl mercaptan. Diversifying the flavor agents of SFC could mimic the complex Maillard reaction of conventional meat in cultured meat systems.

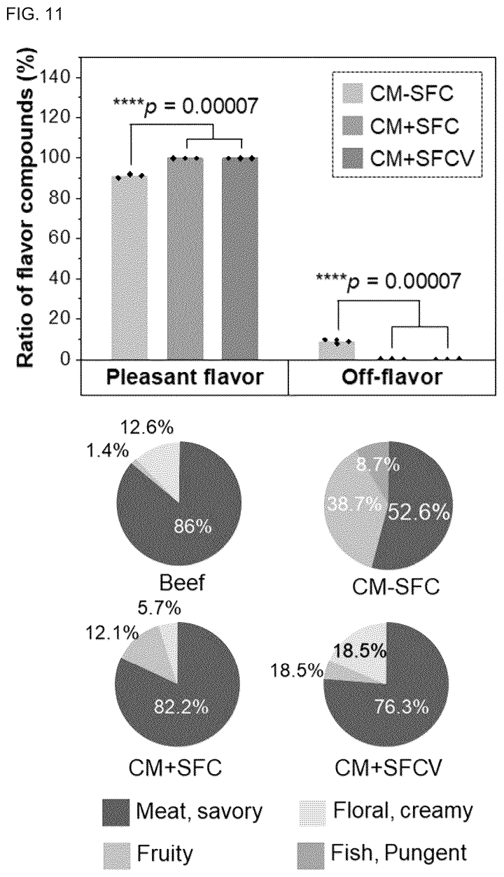

FIG. 11 shows the result of analyzing the ratio of flavor compounds detected in the cultured meat of Comparative Example 3 (CM−SFC), the cultured meat of Example 5 (CM+SFC) and the cultured meat of Example 6 (CM+SFCV), and the pie chart shows the specific flavor profile of each group.
(c) Principal component analysis (PCA) of the flavor compounds in each group (discrimination index=90, n=3). Source data are provided as a source data file.

CM−SFC, CM+SFC and CM+SFCV were cooked at 150° C. for 5 minutes and then flavor evaluation was performed using an electronic nose. After comparing pleasant flavor and off-flavor, the ratio of specific flavor notes was analyzed for each group. Compared to the above biological analysis results, different and more various flavor compounds were detected due to the different polarity of the columns used in the electronic nose. Nonpolar and mild-polar volatile compounds were detected in the electronic nose analysis, whereas polar volatile compounds were detected in the biological analysis. As a result, the volatile compounds with off-flavor (fishy, pungent flavor) which were classified based on the flavor compound classification were detected only from CM−SFC. On the other hand, the volatile compounds with pleasant flavor were detected from all groups, but were detected at high levels from CM+SFC and CM+SFCV. The ratio of the compounds with meaty flavor was higher in CM+SFC and CM+SFCV as compared to CM−SFC. Especially, the flavor characteristic of CM+SFCV was most similar to that of traditional beef among the three cultured meat samples. The flavor ratio of the flavor compounds detected according to the Maillard reaction of beef was high in the following order: meat, floral, creamy and fruity. This pattern was also confirmed in CM+SFCV.

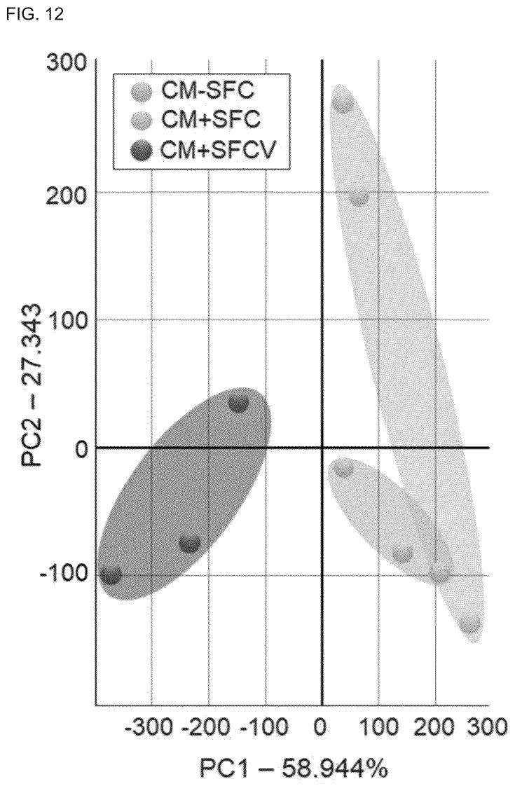

FIG. 12 shows the results of principal component analysis (PCA) to analyze flavor similarity between the cultured meat groups. It was confirmed that SFC and SFCV affect the flavor characteristics of the cultured meat.

In other words, it can be seen that the flavor characteristics of cultured meat can be controlled by introducing switchable flavor compounds (SFCs) into the scaffold for culturing the cultured meat. It can be seen that the complex flavor pattern of real meat can be mimicked by introducing various flavor compounds.

In the present disclosure, robust covalent bonds were introduced to secure the flavor stability during a prolonged open system process for cell proliferation and differentiation. In addition, disulfide bonds were introduced considering temperature responsivity, and the switchable flavor compounds (SFCs) could realize the Maillard reaction of real meat in the cell-cultured meat. The switchable flavor compound (SFC) of the present disclosure remained stable in the scaffold during the prolonged cell culture period, and it released flavor compounds such as furfuryl mercaptan selectively at the cooking temperature. In addition to the flavor compounds included in switchable flavor compound (SFC), more diverse flavor compounds with meaty flavor and savory flavor were found at 150° C. This is due to the hydrogen peroxide, an oxidizing agent for forming disulfide bonds in the Maillard reaction products. The hydrogen peroxide remained in the cultured meat (CM+SFC) of Example 5 at a concentration of 0.386 mM, which is within the food grade concentration range. When furfuryl mercaptan is generated during cooking, the oxidative reactivity of hydrogen peroxide can induce a disulfide bond within the furfuryl mercaptan, forming 2,2-(dithiodimethylene)difuran. Moreover, the hydrogen peroxide could generate hydroxyl or hydroperoxyl radicals upon heating. Hence, these radicals could induce bond scission to form various volatile compounds with meaty and savory flavors, such as 2-methylthiophene or furfuryl methyl disulfide.

In addition, as a result of preparing flavor-enriched cultured meat after performing cell proliferation and differentiation of bovine myoblasts on the switchable flavor compound (SFC)-introduced scaffold, the switchable flavor compound (SFC) exhibited savory and meaty flavors upon volatilization and also generated various flavor molecules by dynamic disulfide exchange. The flavor pattern of cultured meat was similar to that of slaughtered beef.

Although the present disclosure has been described by specific exemplary embodiments and limited examples and comparative examples, they are provided only for a more general understanding of the present disclosure, and the present disclosure is not limited to the examples described above. Those having ordinary knowledge in the art to which the present disclosure belongs can make various changes and modifications based on the above description.

Accordingly, the scope of the present disclosure should not be limited to the described exemplary embodiments, and it should be understood that the scope of the appended claims described below as well as all modifications that are equivalent to the scope of the claims shall fall within the scope of the present disclosure.

What is claimed is:

1. A scaffold for cultured meat, comprising a hydrogel to which a flavor compound comprising a disulfide bond-containing functional group is bound.

2. The scaffold for cultured meat according to claim 1, wherein the disulfide bond-containing functional group is represented by any one selected from Chemical Formulas 1 to 3:

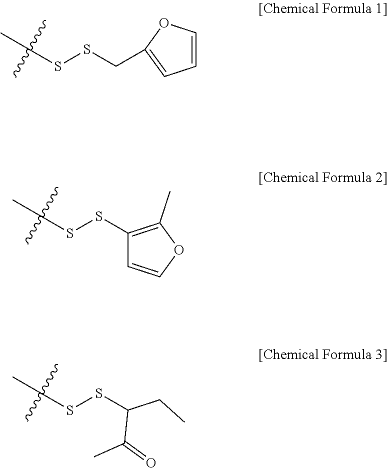

3. The scaffold for cultured meat according to claim 2, wherein the flavor compound is represented by Chemical Formula 4: 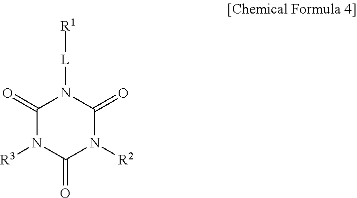
wherein
R¹ is any one of the functional groups represented by Chemical Formulas 1 to 3,
L is (C₁-C₃₀) alkylene, (C₂-C₃₀) heteroalkylene, (C₃-C₃₀) cycloalkylene, (C₂-C₃₀) heterocycloalkylene, (C₆-C₃₀) arylene or (C₅-C₃₀) heteroarylene,
the (C₂-C₃₀) heteroalkylene is one in which each of at least one of a plurality of methylenes (—CH₂—) is independently substituted with —O—, —NH— or —C(═O)—, and
each of R² and R³ is independently a substituent having an acrylic group or a methacryl group bonded to a terminal.

4. The scaffold for cultured meat according to claim 3, wherein the Chemical Formula 4 is represented by the Chemical Formula 5: 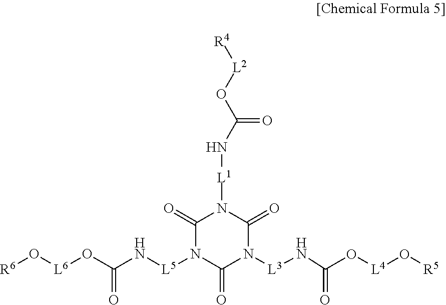
wherein
R⁴ is any one of the functional groups represented by Chemical Formulas 1 to 3, each of R⁵ and R⁶ is an acrylic group or a methacrylic group,
L₁ is (C₁-C₁₅) alkylene,
L₂ is (C₁-C₅) alkylene,
each of L₃ and L₅ is independently (C₁-C₁₅) alkylene, and
each of L₄ and L₆ is independently (C₁-C₅) alkylene.

5. The scaffold for cultured meat according to claim 1, wherein the hydrogel comprises a protein-based polymer.

6. The scaffold for cultured meat according to claim 5, wherein the acrylic or methacrylic group of the flavor compound is copolymerized with the acrylic or methacrylic group side chain of the protein-based polymer to form a hydrogel.

7. The scaffold for cultured meat according to claim 1, wherein the flavor compound is comprised in an amount of 0.1 to 5 wt % based on the total weight of the hydrogel on a dry mass basis.

8. A method for preparing cultured meat, comprising:
filling the scaffold for cultured meat according to claim 1 with a culture medium and inoculating animal cells;
incubating the culture medium inoculated with the animal cells to proliferate the cells; and
organizing the animal cells proliferated in the culture medium.

9. The method for preparing cultured meat according to claim 8, wherein the animal cells comprise animal myoblasts and animal adipocytes.

10. The method for preparing cultured meat according to claim 8, wherein said organizing comprises treating the animal cells with stimulus selected from ultrasound, electric current, electromagnetic field, magnetic field and a combination thereof.

11. Cultured meat prepared by the method for preparing cultured meat according to claim 9.

12. A food composition comprising cultured meat according to claim 11.
